(19) United States
(12) Patent Application Publication
(10) Pub. No.: US 2004/0219626 A1
(43) Pub. Date: Nov. 4, 2004

(54) Method for identifying fungicidally active compounds

(76) Inventors: Martin Vaupel, Leichlingen (DE); Oliver Guth, Leverkusen (DE); Karl-Heinz Kuck, Langenfeld (DE)

Correspondence Address: BAYER CROPSCIENCE LP, Patent Department, 100 BAYER ROAD, PITTSBURGH, PA, 15205-9741, US

(21) Appl. No.: 10/662,908

(22) Filed: Sep. 15, 2003

(30) Foreign Application Priority Data
Sep. 16, 2002 (DE) 10242940.5

(51) Int. Cl. C12Q 1/42; C12Q 1/18

(52) U.S. Cl. 435/21; 435/32

(57) ABSTRACT

The invention relates to a method for identifying fungicides, to the use of farnesyl-pyrophosphate synthase for identifying fungicides, and to the use of farnesyl-pyrophosphate synthase inhibitors as fungicides.

The present application claims priority of German Patent Application Serial No. 102 42 940.5 filed Sep. 16, 2002.

### BACKGROUND OF THE INVENTION

1. Field of the Invention

The invention relates to a method for identifying fungicides, to the use of farnesyl-pyrophosphate synthase for identifying fungicides, and to the use of farnesyl-pyrophosphate synthase inhibitors as fungicides.

2. Description of the Related Art

Undesired fungal growth which leads every year to considerable damage in agriculture can be controlled by the use of fungicides. The demands made on fungicides have increased constantly with regard to their activity, costs and, above all, ecological soundness. There exists therefore a demand for new substances or classes of substances which can be developed into potent and ecologically sound new fungicides. In general, it is customary to search for such new lead structures in greenhouse tests. However, such tests require a high input of labour and a high financial input. The number of the substances which can be tested in the greenhouse is, accordingly, limited. An alternative to such tests is the use of what are known as high-throughput screening (UTS) methods. This involves testing a large number of individual substances with regard to their effect on cells, individual gene products or genes in an automated method. When certain substances are found to have an effect, they can be studied in conventional screening methods and, if appropriate, developed further.

Advantageous targets for fungicides are frequently searched for in essential biosynthetic pathways. Ideal fungicides are, moreover, those substances which inhibit gene products which have a decisive importance in the manifestation of the pathogenicity of a fungus.

### SUMMARY OF THE INVENTION

It was therefore an aim of the present invention to identify, and make available, a suitable new target for potential fungicidal active compounds and to provide a method, based thereon, which makes possible the identification of modulators of this target and thus eventually to provide novel fungicides.

More particularly, the present invention is directed to a method of identifying fungicides, characterized in that a chemical compound is assayed in a farnesyl-pyrophosphate synthase inhibition assay.

More particularly still, the present invention is directed to a method of identifying fungicides, characterized in that

(a) a host cell which expresses a sufficient amount of a farnesyl-pyrophosphate synthase or a polypeptide with the enzymatic activity of a farnesyl-pyrophosphate synthase is brought into contact with a chemical compound or a mixture of chemical compounds under conditions which permit the interaction of the chemical compound with the polypeptide,

(b) the farnesyl-pyrophosphate synthase activity in the absence of a chemical compound is compared with the farnesyl-pyrophosphate synthase activity in the presence of a chemical compound or a mixture of chemical compounds, and

(c) the chemical compound which specifically inhibits farnesyl-pyrophosphate synthase is identified. The present invention is also directed to the use of polypeptides with the activity of a farnesyl-pyrophosphate synthase for identifying fungicides, to the use of inhibitors of polypeptides with the activity of a farnesyl-pyrophosphate synthase as fungicides, and to the use of inhibitors of polypeptides with the activity of a farnesyl-pyrophosphate synthase, which inhibitors are identified by the above described method. The present invention is also directed to fungicidal compounds found in the method described above and to the use of such compounds in the preparation of fungicidal compositions.

### DESCRIPTION OF THE FIGURES

FIG. 1 illustrate the farnesyl-pyrophosphate synthase catalysis of the reaction of dimethylallyl pyrophosphate and isopentenyl pyrophosphate to pyrophosphate and geranyl pyrophosphate, to which a further isopentenyl pyrophosphate molecule is subsequently transferred. A total of two pyrophosphate molecules are liberated in the reaction.

FIGS. 2A and 2B illustrates the homology between farnesyl-pyrophosphate synthases from a variety of fungi: (1) Saccharomycs cerevisiae (S.c.), (2) Neurospora crassa (N.c.), (3) Schizosaccharomycespombe (S.p.), (4) Gibberella fujikuroi (G.f.), (5) Kluyveromyces lactis (K.l.), (6) Claviceps purpurea (C.p.), and (7) Sphaceloma manihoticola (S.m.). The frames represent regions whose sequences are exactly the same (consensus sequence).

FIG. 3 illustrates the heterologous expression of farnesyl-pyrophosphate synthase in E. coli Origami. The overexpressed GST fusion protein is 65 kDa in size. A size marker was applied in lane M. Lane 1: purified farnesyl-pyrophosphate synthase; lane 2: cytoplasm fraction of the overexpressed farnesyl-pyrophosphate synthase 4 hours after induction with IPTG; lanes 2 and 3: wash fractions after application of the cytoplasm fraction to the glutathione-Sepharose column; lane 4: elution fraction with purified farnesyl-pyrophosphate synthase.

FIG. 4 illustrates the kinetics of the conversion of dimethylallyl pyrophosphate and isopentenyl pyrophosphate by different concentrations of farnesyl-pyrophosphate synthase in the assay. 42 μM isopentenyl pyrophosphate, 54 μM dimethylallyl pyrophosphate, 0.34 mU inorganic pyrophosphatase and 0.05 μg of farnesyl-pyrophosphate synthase were employed in an assay volume of 40 μl. The protein concentrations used, of farnesyl-pyrophosphate synthase, can be seen from the figure. The conversion was monitored with reference to the increase in absorption at 620 nm on the basis of the reaction of the liberated orthophosphate with malachite green solution.

FIG. 5 illustrates the determination of the K_{M} value for dimethylallyl pyrophosphate. Lineweaver/Burk representation of the data: 1/Vₒ=1/Vₘₐₓ+1/S×(K_{M}/Vₘₐₓ), where Vₒ is the initial reaction rate, Vₘₐₓ the maximum reaction rate possible and S the substrate concentration. Vₘₐₓ and K_{M} can then be read as the intercepts on the horizontal and the vertical axes 1/Vₘₐₓ and 1/K_{M}, respectively.

FIG. 6 illustrates the determination of the K_{M} value for isopentenyl pyrophosphate. Lineweaver/Burk representation of the data: 1/Vₒ=1/Vₘₐₓ+1/S×(K_{M}/Vₘₐₓ), where Vₒ is the initial reaction rate, Vₘₐₓ the maximum reaction rate possible and S the substrate concentration. Vₘₐₓ and K_{M} can then be read as the intercepts on the horizontal and the vertical axes 1/Vₘₐₓ and 1/K_{M}, respectively.

### DETAILED DESCRIPTION OF THE INVENTION

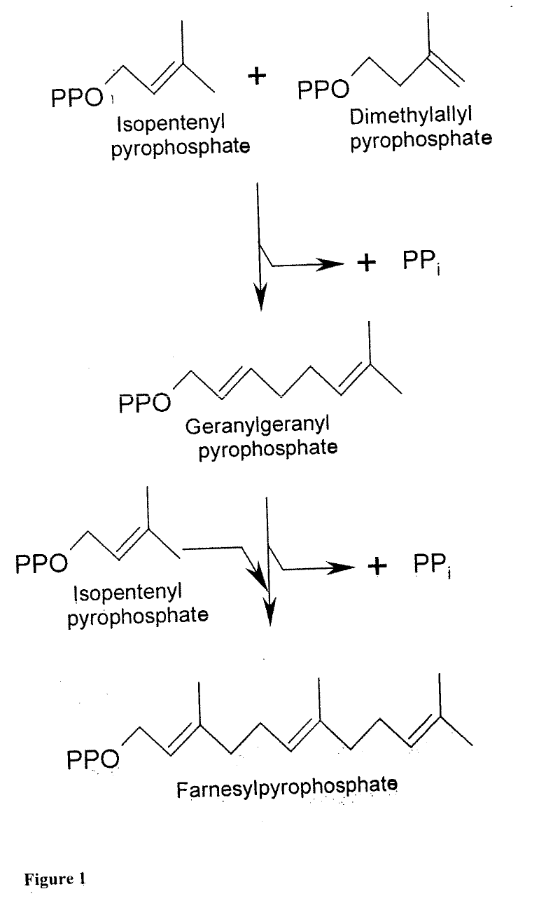

Farnesyl-pyrophosphate synthase (EC 2.5.1.1 and 2.5.1.10), also known as farnesyl-pyrophosphate synthetase, farnesyl-diphosphate synthase or farnesyl-diphosphate synthetase, initially catalyzes the reaction of dimethylallyl pyrophosphate and isopentenyl pyrophosphate to pyrophosphate and geranyl pyrophosphate (dimethylallyltransferase reaction; EC 2.5.1.1). A further isopentenyl pyrophosphate molecule is subsequently transferred to geranyl pyrophosphate, giving rise to farnesyl pyrophosphate and pyrophosphate (geranyl transtransferase reaction; EC 2.5.1.10) (FIG. 1).

The reactions catalyzed by farnesyl-pyrophosphate synthase are essential steps for providing farnesyl pyrophosphate for ergosterol, dolichol or ubiquinone biosynthesis (Lees et al., 1997, Biochemistry and molecular biology of sterol synthesis in Saccharomyces cerevisiae. Biochemistry and Function of Sterols, 85-99; Mercer, 1984, The biosynthesis of ergosterol. Pestic. Sci. 15(2), 133-55; Szkopinska et al., 1997, Polyprenol formation in the yeast Saccharomyces cerevisiae: effect of farnesyl diphosphate synthase overexpression. Journal of Lipid Research 38(5), 962-968).

Genes for farnesyl-pyrophosphate synthase have been cloned from a variety of fungi, namely from the Ascomycetes Saccharomyces cerevisiae (Swissprot Accession No.: P08524), Schizosaccharomyces pombe (Swissprot Accession No.: O14230) and Neurospora crassa (Swissprot Accession No.: Q92250) and from the phytopathogenic fungus Gibberella fujikuroi (Swissprot Accession No.:

Q92235). Sequence fragments are also known from the phytopathogenic fungi Claviceps purpurea and Sphaceloma manihoticola. In addition, farnesyl-pyrophosphate synthase has also been obtained from a large number of other organisms, for example from Homo sapiens (Swissprot: Accession No.: P14324), tomato (Swissprot: Accession No.:O 65004) or maize (Swissprot: Accession No.: P493 53).

The sequence similarities are significant within the classes of the eukaryotes, while the sequence identity with the bacterial enzymes is less significant.

Farnesyl-pyrophosphate synthase has been isolated-for example from yeast, expressed, purified and characterized (Anderson et al., 1989, Farnesyl diphosphate synthetase. Molecular cloning, sequence, and expression of an essential gene from Saccharomyces cerevisiae. J Biol. Chem. 264(32), 19176-84; Eberhardt et al., 1975, Prenyltransferase from Saccharomyces cerevisiae. Purification to homogeneity and molecular properties. Journal of Biological Chemistry 250(3), 863-6; Song et al., 1994, Yeast farnesyl-diphosphate synthase: Site-directed mutagenesis of residues in highly conserved prenyltransferase domains I and II. Proc. Natl. Acad. Sci. U.S.A. 91(8), 3044-8).

The term “identity” as used in the present context refers to the number of sequence positions that are identical in an alignment. In most cases, it is indicated as a percentage of the alignment length.

The term “similarity” as used in the present context, in contrast, assumes the existence of a similarity metric, that is to say a measure for the desired assumed similarity, for example, between a valine and a threonine or a leucine.

The term “homology” as used in the present context, in turn, indicates evolutionary relationship. Two homologous proteins have developed from a shared precursor sequence. The term is not necessarily about identity or similarity, apart from the fact that homologous sequences usually have a higher degree of similarity (or occupy more identical positions in an alignment) than non-homotogous sequences.

The term “complete farnesyl-pyrophosphate synthase” as used in the present context describes farnesyl-pyrophosphate synthase encoded by the complete coding region of a transcription unit, starting with the ATG start codon and comprising all the information-bearing exon regions of the gene encoding farnesyl-pyrophosphate synthase which is present in the source organism, as well as the signals required for correct transcriptional termination.

The term “biological activity of a farnesyl-pyrophosphate synthase” as used in the present context refers to the ability of a polypeptide to catalyse the above-described reaction, i.e. the conversion of dimethylallyl pyrophosphate and isopentenyl pyrophosphate into farnesyl-pyrophosphate.

The term “active fragment” as used in the present context describes nucleic acids encoding farnesyl-pyrophosphate synthase which are no longer complete, but still encode polypeptides with the biological activity of a farnesyl-pyrophosphate synthase and which are capable of catalysing a reaction characteristic of farnesyl-pyrophosphate synthase, as described above. Such fragments are shorter than the above-described complete nucleic acids encoding farnesyl-pyrophosphate synthase. In this context, nucleic acids may have been removed both at the 3′ and/or 5′ ends of the sequence, or else parts of the sequence which do not have a decisive adverse effect on the biological activity of farnesyl-pyrophosphate synthase may have been deleted, i.e. removed. A lower or else, if appropriate, an increased activity which still allows the characterization or use of the resulting farnesyl-pyrophosphate synthase fragment is considered as sufficient for the purposes of the term as used herein. The term “active fragment” may likewise refer to the amino acid sequence of farnesyl-pyrophosphate synthase in this case, it applies analogously to what has been said above for those polypeptides which no longer contain certain portions in comparison with the above-described complete sequence, but where no decisive adverse effect is exerted on the biological activity of the enzyme. The fragments may differ with regard to their length.

The term “gene” as used in the present context is the name for a segment from the genome of a cell which is responsible for the synthesis of a polypeptide chain.

The term “fungicide” or “fungicidal” as used in the present context refers to chemical compounds which are suitable for controlling fungi, in particular phytopathogenic fungi. Such phytopathogenic fungi are mentioned hereinbelow, the enumeration not being final:

Plasmodiophoromycetes, Oomycetes, Chytridiomycetes, Zygomycetes, Ascomycetes, Basidiomycetes and Deuteromycetes, for example

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries; Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotrichoides.

Others which are of particular interest are, for example, Magnaporthe grisea, Cochliobulus heterosirophus, Nectria hematococcus and Phytophthora species.

The present invention therefore also relates to a method for identifying fungicides, i.e. farnesyl-pyrophosphate synthase inhibitors from phytopathogenic fungi, which can be used as fungicides for controlling fungal attack in plants.

However, fungicidal active substances which are found with the aid of the farnesyl-pyrophosphate synthase according to the invention, can also interact with farnesyl-pyrophosphate synthase from fungal species which are pathogenic for humans, it not being necessary for the interaction with the different farnesyl-pyrophosphate synthases which occur in these fungi to be always equally pronounced.

The invention therefore relates to a method for identifying antimycotics, i.e. farnesyl-pyrophosphate synthase inhibitors from fungi which are pathogenic for humans or animals, for the preparation of compositions for the treatment of diseases caused by fungi which are pathogenic for humans or animals.

Of particular interest in this context are, the following fungi which are pathogenic to humans and which may cause, amongst others, the symptoms stated hereinbelow:

Dermatophytes such as, for example, Trichophyton spec., Microsporum spec., Epidermophytonfloccosum or Keratomyces ajelloi, which cause, for example, Athlete's foot (Tinea pedis),

Yeasts such as, for example, Candida albicans, which causes soor oesophagitis and dermatitis, Candida glabrata, Candida krusei or Cryptococcus neoformans, which may cause, for example, pulmonal cryptococcosis or else torulosis,

Moulds such as, for example, Aspergillus fumigatus, A. flavus, A. niger, which cause, for example, bronchopulmonary aspergillosis or fungal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella mycetomatis, which causes, for example, subcutaneous mycetomas, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

The terms “fungicidal” or “fungicide” will be used hereinbelow equally for the terms “antimycotic” and for the terms “fungicidal” or “fungicide” in the traditional sense, i.e. referring to phytopathogenic fungi.

Fungicidal active substances which can be found with the aid of a farnesyl-pyrophosphate synthase obtained from a specific fungus, in the present case for example from S. cerevisiae, can therefore also interact with a farnesyl-pyrophosphate synthase from a large number of Qther fungal species, in particular also with phytopathogenic fungi, it not always being necessary for the interaction with the different farnesyl-pyrophosphate synthases which occur in these fungi to be equally pronounced. This explains, inter alia, the selectivity which has been observed in the substances which act on this enzyme.

The term “competitor” as used in the present context refers to the property of the cornpounds to compete with other, possibly yet to be identified, compounds for binding to farnesyl-pyrophosphate synthase and to displace the latter, or to be displaced by the latter, from the enzyme.

The term “agonist” as used in the present context refers to a molecule which accelerates or increases the farnesyl-pyrophosphate synthase enzyme activity.

The term “antagonist” as used in the present context refers to a molecule which slows down or prevents the farnesyl-pyrophosphate synthase enzyme activity.

The term “modulator” as used in the present context is the generic term for agonist or antagonist. Modulators can be small organochemical molecules, peptides or antibodies which bind to the polypeptides according to the invention or influence their activity. Moreover, modulators can be small organochemical molecules, peptides or antibodies which bind to a molecule which, in turn, binds to the polypeptides according to the invention, thus influencing their biological activity. Modulators can be natural substrates and ligands, or structural or functional mimetics of these. However, the term “modulator” as used in the present context takes the form of those molecules which do not constitute the natural substrates or ligands.

Despite extensive research into farnesyl-pyrophosphate synthase, it was hitherto unknown that farnesyl-pyrophosphate synthase may constitute, in fungi, a target protein (what is known as “target”) for fungicidally active substances.

No fungicidal action has been described for prior-art farnesyl-pyrophosphate synthase inhibitors (see, for example, Bergstrom et al., 2000, Alendronate Is a Specific, Nanomolar Inhibitor of Farnesyl Diphosphate Synthase. Archives of Biochemistry and Biophysics 373(1), 231-241; Dunford et al., 2001, Structure-activity relationships for inhibition of farnesyl diphosphate synthase in vitro and inhibition of bone resorption in vivo by nitrogen-containing bisphosphonates. Journal of Pharmacology and Experimental Therapeutics 296(2), 235-242; Thompson et al., 2002, Identification of a Bisphosphonate That Inhibits Isopentenyl Diphosphate Isomerase and Farnesyl Diphosphate Synthase. Biochemical and Biophysical Research Communications 290(2), 869-873).

The present invention now shows for the first time that farnesyl-pyrophosphate synthase constitutes an enzyme which is important in particular for fungi and which is therefore particularly suitable as target protein for the search for further, and improved, fungicidally active substances. The present invention furthermore demonstrates that the enzyme farnesyl-pyrophosphate synthase furthermore suits methods for identifying modulators or inhibitors of the enzyme acivity of the polypeptide in suitable assays, which is not always the case in various targets which are of theoretic interest.

It is furthermore shown within the scope of present invention that farnesyl-pyrophosphate synthase can indeed be influenced by active substances, and that inhibition of the fungal farnesyl-pyrophosphate synthase leads to damage or the death of the treated fungus.

Thus, a method was developed within the scope of the present invention which is suitable for determining the enzyme activity of farnesyl-pyrophosphate synthase and the inhibition of this activity by one or more substances in what is known as an inhibition assay, thus identifying inhibitors of the enzyme, for example in HTS and UHTS methods. Inhibitors which have been identified in vitro can then be tested in vivo for their fungicidal activity.

It has furthermore been found within the scope of the present invention that farnesyl-pyrophosphate synthase can also be inhibited in vivo by active substances, and that a fungal organism which is treated with these active substances can be damaged or destroyed by the treatment of these active substances. The inhibitors of a fungal farnesyl-pyrophosphate synthase can thus be used as fungicides, in particular in crop protection, or else as antimycotics for pharmaceutical indications. For example, it is demonstrated in the present invention that inhibition of farnesyl-pyrophosphate synthase with one of the substances identified in a method according to the invention leads to growth inhibition or to the death of the treated fungi in synthetic media or on the plant.

A farnesyl-pyrophosphate synthase which can be employed in a method according to the invention can be obtained, for example, from fungi such as S. cerevisiae. To prepare the yeast farnesyl-pyrophosphate synthase, it is possible, for example, to express the gene recombinantly in Escherichia coli and to prepare an enzyme preparation from E. coli cells (Example 1).

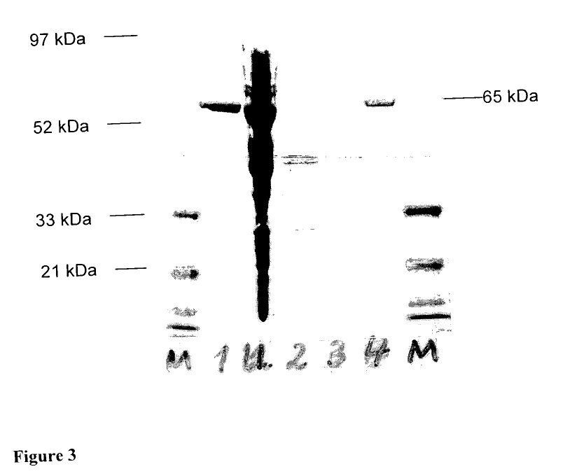

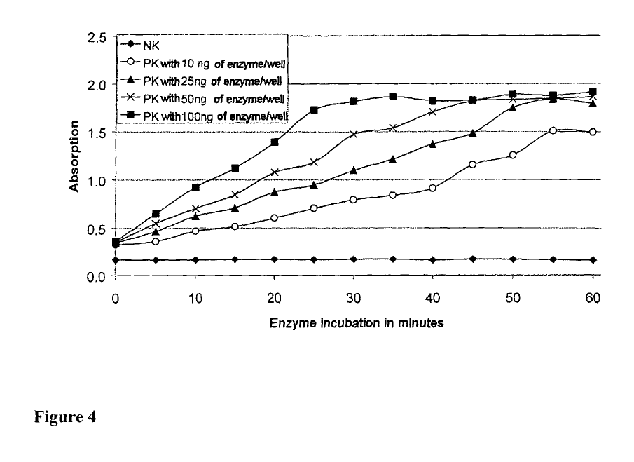

To express the polypeptide Erg20, which is encoded by erg20, (Chambon et al., 1990, Isolation and properties of yeast mutants affected in farnesyl diphosphate synthetase. Curr. Genet. 18(1), 41-6; SWISS-PROT Accession Number: P08524), the corresponding ORF was amplified from genomic DNA by methods known to the skilled worker using gene-specific primers. The DNA in question was cloned into the vector pGEX-4T-1 (Pharmacia Biotech, makes possible the introduction of an N-terminal GST tag). The resulting plasmid pErg20 contains the complete coding sequence of erg20 in N-terminal fusion with a GST tag from the vector. The Erg20 fusion protein has a calculated mass of 64.5 kDa (cf. Example 1 and FIG. 3).

Plasmid pErg20 was then used for the recombinant expression of Erg20 in E. coli Origami cells (cf. Example 1).

As already explained above, the present invention is not only restricted to the use of yeast farnesyl-pyrophosphate synthase. Polypeptides with the activity of a farnesyl-pyrophosphate synthase can also be obtained analogously from other fungi, preferably from phytopathogenic fungi, in a manner known to the skilled worker, and these polypeptides can then be employed in a method according to the invention. It is preferred to use the S. cerevisiae farnesyl-pyrophosphate synthase.

The term “polypeptides” as used in the present context refers not only to short amino acid chains which are generally referred to as peptides, oligopeptides or oligomers, but also to longer amino acid chains which are normally referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP ribosylations, amidations, covalent linkages to flavins, haem moieties, nucleotides or nucleotide derivatives, lipids or lipid derivatives or phophatidylinositol, cyclizations, disulphide bridge formations, demethylations, cystine formations, formylations, gamma-carboxylations, glycosylations, hydroxylations, iodinations, methylations, myristoylations, oxidations, proteolytic processings, phosphorylations, selenoylations and tRNA-mediated amino acid additions.

The polypeptides according to the invention may exist in the form of “mature” proteins or as parts of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in the source organism, from which they can be obtained directly, for example. Likewise, active fragments of a farnesyl-pyrophosphate synthase may be employed in the methods according to this invention, as long as they make possible the determination of the enzyme activity of the polypeptide, or its inhibition by a candidate compound.

In comparison with the corresponding regions of naturally occurring farnesyl-pyrophosphate synthases, the polypeptides according to the method of the invention can have deletions or amino acid substitutions, as long as they still exert at least the biological activity of a complete farnesyl-pyrophosphate synthase. Conservative substitutions are preferred. Such conservative substitutions encompass variations, one amino acid being replaced by another amino acid from among the following group:

1. Small, aliphatic residues, non-polar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic non-polar residues: Met, Leu, Ile, Val and Cys; and

5. Aromatic residues: Phe, Tyr and Trp.

One possible farnesyl-pyrophosphate synthase purification method is based on preparative electrophoresis, FPLC, HPLC (for example using gel filtration columns, reversed-phase columns or mildly hydrophobic columns), gel filtration, differential precipitation, ion-exchange chromatography or affinity chromatography (cf. Example 1).

A rapid method of isolating the farnesyl-pyrophosphate synthases which are synthesized by host cells starts with expressing a fusion protein, where the fusion moiety may be purified in a simple manner by affinity purification. For example, the fusion moiety may be a GST tag (cf. Example 1), in which case the fusion protein can be purified on a glutathione-Sepharose column. The fusion moiety can be removed by partial proteolytic cleavage, for example at linkers between the fusion moiety and the polypeptide according to the invention which is to be purified. The linker can be designed in such a way that it includes target amino acids, such as arginine and lysine residues, which define sites for trypsin cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, particularly preferably at least 100 times, higher than in a host cell preparation.

The polypeptides according to the invention may also be affinity-purified without fusion moieties with the aid of antibodies which bind to the polypeptides. The method for preparing polypeptides with farnesyl-pyrophosphate synthase activity, such as, for example, the polypeptide Erg20, thus comprises

(a) culturing a host cell containing at least one expressible nucleic acid sequence encoding a polypeptide from fungi with the biological activity of a farnesyl-pyrophosphate synthase under conditions which ensure the expression of this nucleic acid, or

(b) expressing an expressible nucleic acid sequence encoding a polypeptide from fungi with the biological activity of a farnesyl-pyrophoshate synthase in an in-vitro system, and

(c) recovering the polypeptide from the cell, the culture medium or the in-vitro system.

The cells thus obtained which contain the polypeptide according to the invention, or the purified polypeptide thus obtained, are suitable for use in methods for identifying farnesyl-pyrophosphate synthase modulators or inhibitors.

The present invention thus also relates to the use of polypeptides from fungi which exert at least one biological activity of a farnesyl-pyrophosphate synthase in methods for identifying inhibitors of a polypeptide from fungi with the activity of a farnesyl-pyrophosphate synthase, it being possible to use the farnesyl-pyrophosphate synthase inhibitors as fungicides. The S. cerevisiae farnesyl-pyrophosphate synthase is especially preferably used.

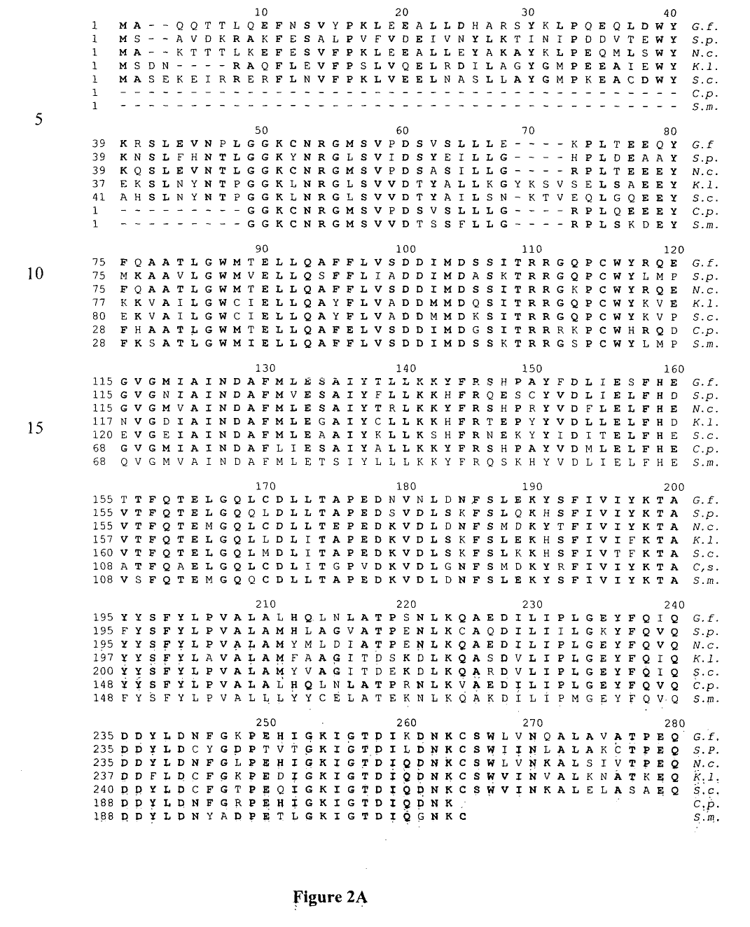

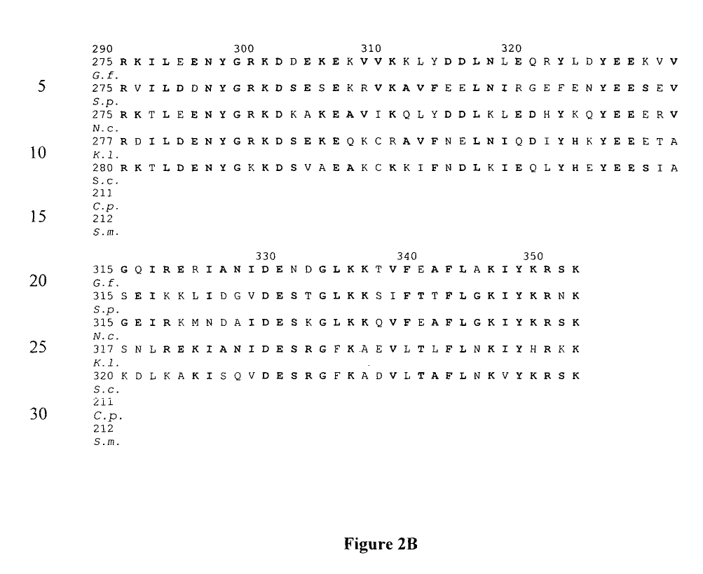

Fungicides which are found with the aid of a farnesyl-pyrophosphate synthase from specific fungal species can thus also interact with farnesyl-pyrophosphate synthases from other fungal species, but the interaction with the different farnesyl-pyrophosphate synthases which are present in these fungi need not always be equally pronounced. This explains inter alia the selectivity of active substances. The fungicidal use in other fungal species of active substances which have been found with a farnesyl-pyrophosphate synthase of a specific fungal species can be attributed to the fact that farnesyl-pyrophosphate synthases from different fungal species are very closely related and show pronounced homology over substantial regions. Thus, it is clear from FIG. 2 that such a homology over substantial sequence segments exists between S. cerevisiae, N. crossa, S. pombe, K. lactis, S. manihoticola, C. purpurea and G. fujikuroi and that, therefore, the effect of the substances found with the aid of yeast farnesyl-pyrophosphate synthase is not limited to S. cerevisiae. Methods of identifying fungicides therefore preferably employ polypeptides with the enzymatic activity of a farnesyl-pyrophosphate synthase which have a consensus sequence as shown in FIG. 2.

The present invention therefore also relates to a method for identifying fungicides by assaying potential inhibitors or modulators of the enzyme activity of farnesyl-pyrophosphate synthase (candidate compound) in a farnesyl-pyrophosphate synthase inhibition assay.

Methods which are suitable for identifying modulators, i.e. in particular inhibitors or antagonists, of the polypeptides according to the invention are generally based on the determination of the activity or the biological functionality of the polypeptide. Suitable for this purpose are, in principle, methods based on intact cells (in-vivo methods), but also methods which are based on the use of the polypeptide isolated from the cells, which may be present in purified or partially purified form or else as a crude extract. These cell-free in-vitro methods, like in-vivo methods, can be used on a laboratory scale, but preferably also in HTS or UHTS methods. Following the in-vivo or in-vitro-identification of modulators of the polypeptide, fungal cultures can be assayed in order to test the fungicidal activity of the compounds which have been found.

A large number of assay systems for the purpose of assaying compounds and natural extracts are preferably designed for high throughput numbers in order to maximize the number of substances assayed within a given period. Assay systems based on cell-free processes require purified or semipurified protein. They are suitable for an “initial” assay, which aims mainly at detecting a possible effect of a substance on the target protein. Once such an initial assay has taken place, and one or more cornpounds, extracts and the like have been found, the effect of such compounds can be studied in the laboratory in a more detailed fashion. Thus, inhibition or activation of the polypeptide according to the invention in vitro can be assayed again as a first step in order to subsequently assay the activity of the compound on the target organism, in this case one or more phytopathogenic fungi. If appropriate, the compound can then be used as starting point for the further search and development of fungicidal compounds which are based on the original structure, but are optimized with regard to, for example, activity, toxicity or selectivity.

To find modulators, for example a synthetic reaction mix (for example in-vitro transcription products) or a cellular component such as a membrane, a compartment or any other preparation containing the polypeptides according to the invention can be incubated together with an optionally labelled substrate or ligand of the polypeptides in the presence and absence of a candidate molecule which can be an antagonist. The ability of the candidate molecule to inhibit the activity of the polypeptidcs according to the invention canibe identified for example on the basis of reduced binding of the optionally labelled ligand or a reduced conversion of the optionally labelled substrate. Molecules which inhibit the biological activity of the polypeptides according to the invention are good antagonists.

Detection of the biological activity of the polypeptides according to the invention can be improved by what is known as a reporter system. In this aspect, reporter systems comprise, but are not restricted to, calorimetrically or fluorimetrically detectable substrates which are converted into a product, or a reporter gene which responds to changes in the activity or the expression of the polypeptides according to the invention, or other known binding assays.

A further example of a method by which modulators of the polypeptides according to the invention can be found is a displacement assay in which the polypeptides according to the invention and a potential modulator are combined, under suitable conditions, with a molecule which is known to bind to the polypeptides according to the invention, such as a natural substrate or ligand or a substrate or ligand mimetic. The polypeptides according to the invention can themselves be labelled, for example fluorimetrically or calorimetrically, so that the number of the polypeptides which are bound to a ligand or which have undergone a conversion can be determined accurately. However, binding can likewise be monitored by means of the optionally labelled substrate, ligand or substrate analogue. The efficacy of an antagonist can be determined in this manner.

Effects such as cell toxicity are, as a rule, ignored in these in-vitro systems. The assay systems check not only inhibitory, or suppressive effects of the substances, but also stimulatory effects. The efficacy of a substance can be checked by concentration-dependent assay series. Control mixtures without test substances can be used for assessing the effects.

Owing to the host cells containing nucleic acids encoding farnesyl-pyrophosphate synthase according to the invention and available with reference to the present invention, the development of cell-based assay systems for identifying substances which modulate the activity of the polypeptides according to the invention, is made possible.

Thus, yet another possibility of identifying substances which modulate the activity of the polypeptides according to the invention is what is known as the scintillation proximity assay (SPA), see EP 015 473. This assay system exploits the interaction of a polypeptide (for example yeast farnesyl-pyrophosphate synthase) with a radiolabelled ligand or substrate. Here, the polypeptide is bound to microspheres or beads which are provided with scintillating molecules. As the radioactivity declines, the scintillating substance in the microsphere is excited by the subatomic particles of the radiolabel, and a detectable photon is emitted. The assay conditions are optimized so that only those particles emitted from the ligand lead to a signal, said particles being emitted by a ligand bound to the polypeptide according to the invention.

The modulators to be identified are preferably small organochemical compounds.

Accordingly, a method for identifying a compound which modulates the activity of a fungal farnesyl-pyrophosphate synthase and which can be used in crop protection as fungicide preferably consists in

a) bringing a polypeptide according to the invention or a host cell containing this polypeptide into contact with a chemical compound or a mixture of chemical compounds under conditions which permit the interaction of a chemical compound with the polypeptide,

b) comparing the activity of the polypeptide according to the invention in the absence of a chemical compound with the activity of the polypeptide according to the invention in the presence of a chemical compound or a mixture of chemical compounds, and

c) identifying the chemical compound which specifically modulates the activity of the polypeptide according to the invention, and, if appropriate,

d) subjecting the fungicidal activity of the compound identified to in-vivo tests.

In this context, the compound which specifically inhibits the activity of the polypeptide according to the invention is particularly preferably determined. The term “activity” as used in the present context refers to the biological activity of the polypeptide according to the invention.

A preferred method exploits the fact that two pyrophosphate molecules are liberated in the farnesyl-pyrophosphate synthase reaction. The activity, or the decrease or increase in activity, of the polypeptide according to the invention can thus be determined by enzymatically cleaving the pyrophosphate by means of inorganic pyrophosphatase and subsequently detecting the orthophosphate which has been liberated, using a phosphate detection reagent. The lower, or inhibited, activity of the polypeptide according to the invention is monitored with reference to the photospectrometric determination of the decrease or increase, of the orthophosphate which has been liberated. The concentration of phosphate which has been liberated can then be determined with a phosphate detection reagent at an absorption maximum at 620 nm.

The measurement can also be carried out in formats conventionally used for HTS or UHTS assays, for example in microtitre plates, into which for example a total volume of 5 to 50 μl is introduced per reaction or per well and the individual components are present in one of the above-stated final concentrations (cf. Example 2). The compound (candidate molecule) to be assayed and which potentially inhibits or activates the activity of the enzyme is introduced for example in a suitable concentration in the above-stated assay buffer, which contains dimethylallyl pyrophosphate and isopentenyl pyrophosphate. The polypeptide according to the invention is then added in the abovementioned assay buffer containing the auxiliary enzyme inorganic pyrophosphatase, which is required for the coupled assay, thus starting the reaction. The mixture is then incubated for example for up to 40 minutes at a suitable temperature, and the increase in absorption is measured at 620 nm.

A further measurement is carried out in a corresponding mixture, but without addition of a candidate molecule and without addition of a polypeptide according to the invention (negative control). Another measurement, in turn, is carried out in the absence of a candidate molecule, but in the presence of the polypeptide according to the invention (positive control). The negative and the positive controls thus provide the reference values for the mixtures in the presence of a candidate molecule.

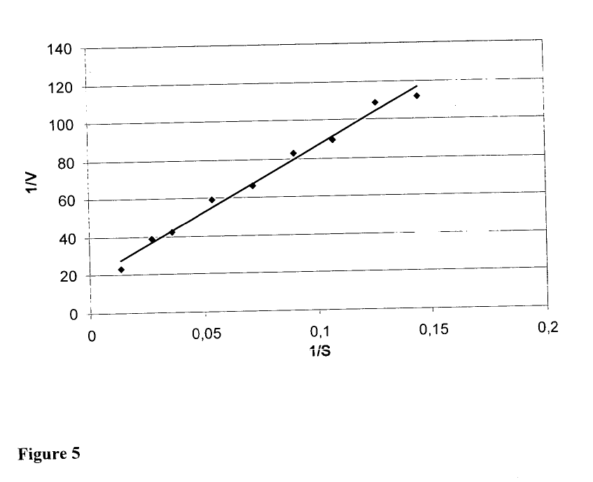

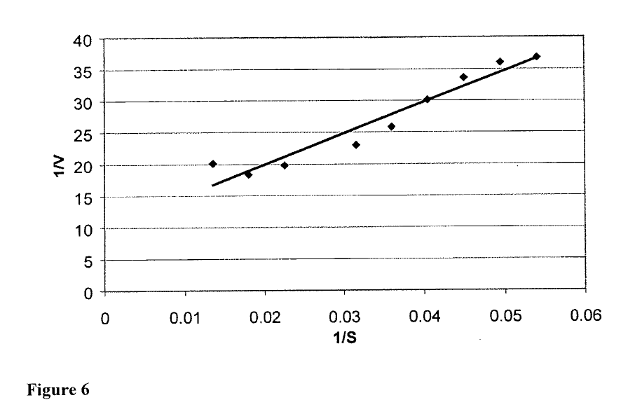

To determine optimal conditions for a method for identifying farnesyl-pyrophos-phate synthase inhibitors or for determining the activity of the polypeptides according to the invention, it may be advantageous to determine the K_{M} value of the polypeptide according to the invention used. This value provides information on the concentration of the substrate(s) to be used by preference. In the case of yeast farnesyl-pyrophosphate synthase, a K_{M} of 36 μM was determined for dimethylallyl pyrophosphate and a K_{M} of 49 μM for isopentenyl pyrophosphate (FIG. 5 and 6).

Compounds which inhibit fungal farnesyl-pyrophosphate synthase and which are capable of damaging (for example inhibiting the growth of) or destroying different fungal species were identified within the scope of the present invention with the aid of the methods which have been described above by way of example.

In addition to the abovementioned methods for determining the enzyme activity of a farnesyl-pyrophosphate synthase or the inhibition of this activity and for identifying fungicides, other methods or inhibitory tests, for example methods or inhibitory tests which are already known, can, of course, also be used as long as they allow the determination of the activity of a farnesyl-pyrophosphate synthase and the detection of an inhibition of this activity by a candidtate compound.

Table I shows examples of compounds which were identified as farnesyl-pyrophosphate synthase inhibitors using the method according to the invention.

The pI50 value shown in this table is the negative decimal logarithm of what is known as the IC50 value which indicates the molar concentration of a substance resulting in 50% inhibition of the enzyme.

A pI50 value of 8, for example, corresponds to half the maximum inhibition of the enzyme at a concentration of 10 nM.

TABLE 1

| Example | Compound | p150 |
|---|---|---|
| 1 | 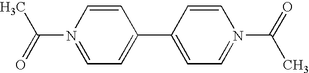 | 5.13 |
| 2 | 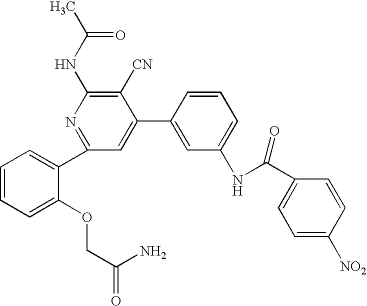 | 4.5 |
| 3 | 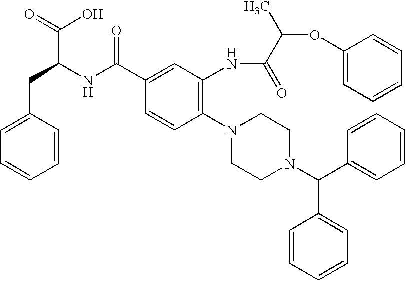 | 4.5 |
| 4 | 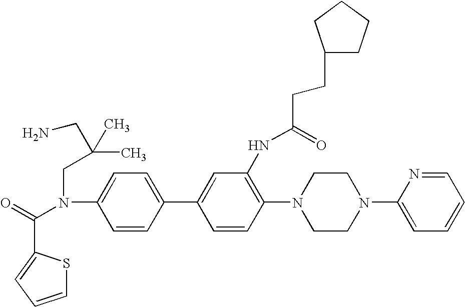 | 4.4 |
| 5 | 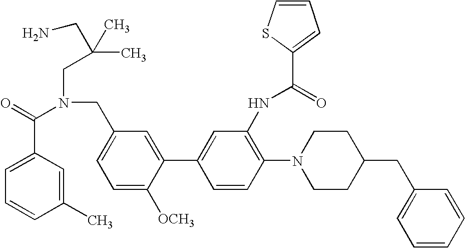 | 4.6 |

It has further been demonstrated within the scope of the present invention that the inhibitors of a farnesyl-pyrophosphate synthase according to the invention which have been identified with the aid of a method according to the invention are capable of damaging or destroying fungi.

To this end, a solution of the active compound to be tested may be pipetted for example into the wells of microtitre plates. After the solvent had evaporated, miedium is added to each well. The miedium is previosly treated with a suitable concentration of spores or mycelia of the test fungus. The resulting concentrations of the active compound are, for example, 0.1, 1, 10 and 100 ppm.

The plates were subsequently incubated on a shaker at a temperature of 22° C. until sufficient growth was discernible in the untreated control.

The plates were evaluated photometrically at a wavelength of 620 nm. The dose of active compound which leads to a 50% inhibition of the fungal growth over the untreated control (ED₅₀) was calculated from the readings of the different concentrations. Table II shows examples of the results of such an assay as ED₅₀ values for compounds found in a method according to the invention (Table I).

The effect, on fungi, of compounds found with the aid of a method according to the invention can also be assayed by testing their protective action for plants. To this end a suitable active substance preparation is prepared. For example 1 part by weight of active substance is mixed with, for example, 24.5 parts by weight of acetone and 24.5 parts by weight of dimethylformamide and 1 part by weight of alkylaryl polyglycol ether as emulsifier, and the concentrate is diluted to the desired concentration.

To test for protective action, young plants are sprayed with the active substance preparation. After the spray coating has dried on, the plants are inoculated with an aqueous suspension (conidia suspension) of a fungus and then remain for 1 day in an incubation chamber at approximately 20° C. and 100% relative atmospheric humidity.

The plants are then placed in a greenhouse at approx. 12° C. and a relative atmospheric humidity of approx. 90%.

The test is evaluated 1 to 12 days after the inoculation. 0% means an efficacy which corresponds to that of the control, while an efficacy of 100% means that no disease is observed.

Table II shows the concentration of various compounds of Table I at which an efficacy of 50% had been achieved in this test. The examples in question can be seen from the fact that an affected plant has been stated.

TABLE II

| Com- | | |
|---|---|---|
| pound | | |
| (Ex.) | Organism | ED₅₀ [ppm] |
| 1 | Botrytis cinerea | 82.18 |
| 1 | Coriolus versicolor | <0.10 |
| 1 | Penicillium brevicaule | 31.62 |
| 2 | Botrytis cinerea | 18.39 |
| 2 | Phytophthora cryptogea | 94.81 |
| 2 | Septoria tritici | 21.17 |
| 3 | Alternaria mali | >100 |
| 3 | Botrytis cinerea | >100 |
| 3 | Phytophthora cryptogea | >100 |
| 3 | Septoria triciti | >100 |
| 3 | Ustilago avenae | >100 |
| 3 | Pyricularia oryzae | >100 |
| 3 | Phytophthora infestans (Plant affected: tomato) | 500 |
| 3 | Phytophthora infestans (Plant affected: barley) | 500 |
| 3 | Aspergillus niger | >100 |
| 3 | Corilus versicolor | >100 |
| 3 | Penicillium brevicaule | >100 |
| 3 | Pseudomonas fluorescens | >100 |
| 4 | Alternaria mali | 4.48 |
| 4 | Botrytis cinerea | 1.96 |
| 4 | Phytophthora cryptogea | 28.15 |
| 4 | Septoria tritici | 1.94 |
| 4 | Ustilago avenae | 3.29 |
| 4 | Pyricularia oryzae | 1.44 |
| 4 | Phytophthora infestans (Plant affected: tomato) | 500 |
| 4 | Phytophthora infestans (Plant affected: barley) | 500 |
| 4 | Aspergillus niger | >100 |
| 4 | Coriolus brevicaule | 1.31 |
| 4 | Penicillium brevicaule | 2.5 |
| 4 | Pseudomonas fluorescens | >100 |
| 5 | Phytophthora infestans (plant affected: tomato) | 548 |
| 5 | Erysiphe graminis (plant affected: barley) | 548 |
| 5 | Pyricularia oryzae (plant affected: rice) | 548 |
| 5 | Leptosphaeria nodorum (plant affected: wheat) | 548 |
| 5 | Alternaria solani (plant affected: tomato) | 548 |
| 5 | Sphaerotheca fuliginea (plant affected: cucumber) | 548 |

The present invention therefore also relates to the use of modulators of fungal farnesyl-pyrophosphate synthase, preferably farnesyl-pyrophosphate synthase froni phytopathogenic fungi, as fungicides.

The present invention also relates to fungicides which have been identified with the aid of a method according to the invention.

Compounds which are identified with the aid of a method according to the invention and which, owing to inhibition of the fungal farnesyl-pyrophosphate synthase, are fungicidally active can then be used for the preparation of fungicidal compositions.

Depending on their respective physical and/or chemical characteristics, the active substances which have been identified can be converted into the customary formulations, such as solutions, emulsions, suspensions, powders, foams, pastes, granules, aerosols, very fine capsules in polymeric substances and in coating compositions for seed and also ULV cold and warm fogging formulations.

These formulations are produced in a known manner, for example by mixing the active compounds with extenders, that is, liquid solvents, liquefied gases under pressure, and/or solid carriers, optionally with the use of surfactants, that is, emulsifiers and/or dispersants, and/or foam formers. In the case of the use of water as an extender, organic solvents can, for example, also be used as cosolvents. As liquid solvents, there are suitable in the main: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, as well as water. By liquefied gaseous extenders or carriers are meant liquids which are gaseous at ambient temperature and under atmospheric pressure, for example aerosol propellants, such as halogenohydrocarbons as well as butane, propane, nitrogen and carbon dioxide. As solid carriers there are suitable: for example ground natural minerals, such as kaolins, clays, talc, chalk, quartz, attapulgite, inontmorillonite or diatomaceous earth, and ground synthetic minerals, such as highly disperse silica, alumnina and silicates. As solid carriers for granules there are suitable: for example crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks. As emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates as well as protein hydrolysates. As dispersants there are suitable: for example lignin-sulphite waste liquors and methylcellulose.

Adhesives such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids can be used in the formulations. Further additives may be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95 per cent by weight of active substance, preferably between 0.5 and 90%.

The active substances according to the invention, as such or in their formulations, can also be used as a mixture with known fungicides, bactericides, acaricides, nernaticides or insecticides, for example in order to widen in this way the spectrum of action or to prevent the build-up of resistance. In many cases, synergistic effects are achieved, i.e. the efficacy of the mixture exceeds the efficacy of the individual components.

When employing the compounds according to the inyention as fungicides, the application rates can be varied within substantial ranges, depending on the application.

All plants and plant parts may be treated in accordance with the invention. In the present context, plants are understood as meaning all plants and plant populations, such as desired and undesired wild plants or crop plants (including naturally occurring crop plants). Crop plants may be plants which can be obtained by traditional breeding and optimization methods or by biotechnological and recombinant methods or combinations of these methods, including the transgenic plants and including those plant varieties which are capable, or not capable, of protection by Plant Breeders' Rights. Plant parts are understood as meaning all aerial and subterranean parts and organs of the plants, such as shoot, leaf, flower and root, examples which are mentioned being leaves, needles, stems, stalks, flowers, fruiting bodies, fruits and seeds, but also roots, tubers and rhizomes. The plant parts also include harvested material and vegetative and generative propagation material, for example cuttings, tubers, rhizomes, slips and seeds.

The treatment according to the invention of the plants and plant parts with the active substances is affected directly or by acting on their environment, habitat or store by the customary treatment methods, for example by dipping, spraying, vaporizing, fogging, scattering, brushing on and, in the case of propagation material, in particular seed, furthermore by coating with one or more coats.

The examples which follow illustrate various aspects of the present invention and are not to be construed as limiting.

### EXAMPLES

### Example 1

Cloning, expression and purification of erg20 and Erg20 from Saccharomyces cerevisiae

To clone and express erg20, the ORF from Saccharomyces cerevi siae genomic DNA was amplified using gene-specific primers. The corresponding DNA, an amplicon 1059 bp in length, was inserted into the vector pGEX-4T-1 from

Pharmacia Biotech (intermediate cloning) and subsequently cloned into the BamHI and AhoI cut vector pGEX-4T-1 (Pharmacia Biotech) via the BamHI and Xhol cleavage sites introduced by the primers. The resulting plasmid pErg20contains the complete coding sequence of erg20 in N-terminal fusion with the GST tag, which is part of the vectors. The Erg20 fusion protein has a calculated mass of 64.5 kDa.

For the heterologous expression, the plasmid pErg20 was transformed into E. coli Origami in such a way that the transformation mixture acted directly as preculture in 50 ml of selection medium. These cells were incubated overnight at 37° C. and subsequently diluted 1:25 in selection medium (LB medium supplemented with 100 μg/ml ampicillin). Induction was effected at OD₆₀₀ₙₘ 0.8 -1.0 using 0.5 mM IPTG (final concentration) at 37° C. The cells were harvested after 4 hours' induction and stored at −20° C. They were disrupted by sonication in lysis buffer (50 mM Tris-HCI, pH 7, 1 mM DTT, 1 mM EDTA, 10% glycerol). The cytoplasm fraction obtained by centrifugation (20 min at 4° C., 10,000 g) was used for the isolation of the protein expressed. Purification was effected following the standard protocol of the manufacturer for glutathione-sepharose columns using a sorbitol buffer (100 mM Tris/HCl, pH 7.3; 300 mM sorbitol, 100 mM NaCl, 5 mM MgCl₂). The elution buffer used was 50 mM Tris/HCl pH 8.0 with 10 mM reduced glutathione. The purified protein was treated in the buffer with glycerol (50 mM Tris-HCl pH 8.0, 10 mM glutathione, 10% glycerol) and stored at −80° C. Approximately 2.0 mg of soluble protein were isolated from 250 ml of culture medium, and this protein was used in methods for identifying farnesyl-pyrophosphate synthase modulators.

### Example 2

### Identification of Farnesyl-Pyrophosphate Synthase Modulators in 384-Well-MTPs in a Coupled Assay

384-well microtitre plates from Greiner were used for identifying farnesyl-pyrophosphate synthase modulators.

The negative control was pipetted into the first column. The negative control was composed of 5 μl of assay buffer (50 mM Tris/HCl pH 7.5, 3 mM MgCl₂, 2 mM DTT, 0.01% Tween 20) with 5% DMSO, 20 μl of Mix 1 (50 mM Tris/HCl pH 7.5, 3 mM MgCI₂, 2 mM DTT, 0.01% Tween 20, 42 μM isopentenyl pyrophosphate, 54 μM dimethylallyl pyrophosphate) and 20 μl of assay buffer (50 mM Tris/HCl pH 7.5,3 mM MgCl₂, 2 mM DTT, 0.01% Tween 20) with 0.34 mU inorganic pyrophosphatase.

The positive control was pipetted into the second column. The positive control was composed of 5 μl of assay buffer with 5% of DMSO, 20 μl of Mix 1 (50 mM Tris/HCl pH 7.5, 3 mM MgCl₂, 2 mM DTT, 0.01% Tween 20, 42 μM isopentenyl pyrophosphate, 54 μM dimethylallyl pyrophosphate) and 20 μl of Mix 2 (50 mM Tris/HCl pH 7.5, 3 mM MgCl₂, 2 mM DTT, 0.01% Tween 20, 0.34 mU inorganic pyrophosphatase, 0.05 μg of farnesyl-pyrophosphate synthase).

A test substance in a concentration of 2 μM in DMSO was introduced into the remaining columns, 5 μl of the assay buffer being used for diluting the substance to a volume of. After addition of 20 μl of Mix 1 (50 mM Tris/HCl pH 7.5, 3 mM MgCl₂, 2 mM DTT, 0.01% Tween 20, 42 μM isopentenyl pyrophosphate, 54 μM dimethylallyl pyrophosphate), 20 μl of Mix 2 (50 mM Tris/HCl pH 7.5, 3 mM MgCl₂, 2 mM. DTT, 0.01% Tween 20, 0.34 mU inorganic pyrophosphatase, 0.05 μg of farnesyl-pyrophosphate synthase) were added to initiate the reaction. This was followed by incubation at room temperature for 40 minutes. The reaction was subsequently quenched by addition of 50 μl of malachite green solution (three parts of 0.025% malachite green solution (in water) were mixed with one part of 2% ammonium heptamolybdate solution (in 4 M HCl) and 39 parts of this solution were mixed with one part of 7.5% Tween 20 (in water) immediately prior to testing) and the mixture was incubated for 90 minutes at room temperature. The orthophosphate generated during the reaction was measured by determining the absorption at 620 nm in a Tecan SPECTRAFluor Plus suitable for MTPs.

### Example 3

### Demonstration of the Fungicidal Effect of the Farnesyl-Pyrophosphatase Synthase Inhibitors Identified

The desired quantity of methanolic solution of the active compound identified with the aid of a method according to the invention (Tab. I), treated with an emulsifier, was pipetted into the wells of microtitre plates. After the solvent had evaporated, 200 μl of potato dextrose medium were added to each well. Suitable concentrations of spores or mycelia of the test fungus (see Table II) were previously added to the medium.

The resulting emulsifier concentration was 300 ppm.

The plates were subsequently incubated on a shaker at a temperature of 22° C. until sufficient growth was observed in the untreated control. Evaluation was done photometrically at a wavelength of 620 nm. The dose of active compound which leads to a 50% inhibition of the fungal growth over the untreated control (ED₅₀) is calculated from the readings of the different concentrations (see Table II).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A method of identifying one or more fungicides comprising assaying a chemical compound in a farnesyl-pyrophosphate synthase inhibition assay.

2. The method according to claim 1, wherein
(a) a host cell which expresses a sufficient amount of a farnesyl-pyrophosphate synthase or a polypeptide with the enzymatic activity of a farnesyl-pyrophosphate synthase is brought into contact with said chemical compound or a mixture of chemical compounds under conditions which permit the interaction of the chemical compound with the polypeptide,
(b) the farnesyl-pyrophosphate synthase activity in the absence of the chemical compound or mixture of chemical compounds is compared with the farnesyl-pyrophosphate synthase activity in the presence of said chemical compound or said mixture of chemical compounds, and
(c) the chemical compound or mixture of chemical compounds which specifically inhibits farnesyl-pyrophosphate synthase is identified.

3. The method according to claim 1 or 2 wherein a fungal farnesyl-pyrophosphate synthase is used.

4. The method according to any one of claims 1 to 2 wherein the inhibition of the enzyme activity of the farnesyl-pyrophosphate synthase is measured on the basis of the amount of phosphate group, determined with a phosphate detection reagent.

5. The method according to any one of claims 1 to 2, further comprising the step of assaying the fungicidal action of the chemical compound identified, by bringing said chemical compound into contact with a fungus.

6. A fungicide, said fungicide comprising an inhibitor of a polypeptide with the activity of a farnesyl-pyrophosphate synthase.

7. The fungicide of claim 6, wherein said inhibitor is identified by a method according to any one of claims 1 to 2.

8. A fungicidal composition comprising one or more fungicidal compounds identified by a method according to any one of claims 1 to 2, and an extender and/or a surfactant.

9. A method of identifying fungicides comprising:
(a) providing a host cell which expresses a farnesyl-pyrophosphate synthase or providing an isolated polypeptide with the enzymatic activity of a farnesyl-pyrophosphate synthase;
(b) providing a chemical compound or a mixture of chemical compounds;
(c) admixing the host cell or the isolated polypeptide and the compound or mixture of compounds under conditions which permit the interaction of the chemical compound or mixture of chemical compounds with the host cell or the isolated polypeptide;
(d) providing a control host cell which expresses the farnesyl-pyrophosphate synthase or providing a control isolated polypeptide with the enzymatic activity of a farnesyl-pyrophosphate synthase and which control host cell or control isolated polypeptide is not admixed with the chemical compound or the mixture of chemical compounds;
(e) comparing the result of step (c) with the result of step (d); and
(f) identifying the chemical compound or mixture of chemical compounds which affects the expression of the farnesyl-pyrophosphate synthase from the host cell or the isolated polypeptide compared to the control host cell or the control isolated polypeptide.

10. The method of claim 9 wherein a fungal farnesyl-pyrophosphate synthase or peptide is used.

11. The method according to any one of claims 9 or 10, wherein the chemical compound or mixture of chemical compounds that affects the admixed host cell or isolated polypeptide is assigned a quantitative value compared with the control host cell or the control isolated polypeptide by comparative titration of released phosphate ion in the admixed host cell with the control host cell or the isolated polypeptide compared with the control isolated polypeptide.

12. The method of claim 9 further comprising admixing the identified chemical compound or the mixture of chemical compounds with a fungus.

13. A fungicidal farnesyl-pyrophosphate synthase inhibitor which is a compound of the Formulae A-E or a salt thereof, wherein the compounds of the Formulae A-E are respectively [chemical structure]
